(19) United States
(12) Patent Application Publication
(10) Pub. No.: US 2001/0023341 A1
(43) Pub. Date: Sep. 20, 2001

(54) Absorbent articles having improved fastening system

(75) Inventor: Hamzeh Karami, Lockhaven, PA (US)

Correspondence Address: Stoll, Miskin, Hoffman & Badie, The Empire State Building, Suite 6110, 350 Fifth Avenue, New York, NY, 10118, US

(73) Assignee: First Quality Enterprises, Inc.

(21) Appl. No.: 09/797,334

(22) Filed: Mar. 1, 2001

Related U.S. Application Data
(63) Continuation-in-part of application No. 09/376,282, filed on Aug. 18, 1999.
Application No. 09/376,282 is a continuation-in-part of application No. 09/097,198, filed on Jun. 12, 1998.
Application No. 09/097,198 is a continuation-in-part of application No. 09/149,265, filed on Sep. 8, 1998.

(51) Int. Cl. A61F 13/15

(52) U.S. Cl. 604/385.03; 604/387

(57) ABSTRACT

A T-shaped diaper comprising two portions, a lateral portion (backwaist) having two lateral segments or wings for wrapping around the waist of a wearer of the diaper, and a second vertical portion or insert which is adapted to be passed under the crotch of the wearer and folded thereover. A uniquely designed tape tab located at the outer lateral end of one wing assures more perfect and comfortable fit. Tape tabs are also provided at the distal ends of vertical portion for engagement onto landing zones disposed on the surface of each wing. These tape tabs may have the same general construction as the tape tab on the diaper wing.

### RELATED APPLICATIONS

This application is a continuation-in-part of application Ser. No. 09/376,282 filed Aug. 18, 1999 which is, in turn, a continuation-in-part of application Ser. No. 09/097,198 filed Jun. 12, 1998 which is, in turn, a continuation-in-part of application Ser. No. 09/149,265 filed Sep. 8, 1998.

### FIELD OF THE INVENTION

The present invention relates to absorbent articles such as disposable diapers, infant training pants, adult incontinent briefs and underpants used for absorption and containment of urine and other body exudates. More particularly, this invention relates to a fastening system used in such articles for providing an improved and effective means of detachably securing the front and rear of such articles. In one particular aspect, this invention relates to providing such fasteners in T-shaped briefs.

### BACKGROUND OF THE INVENTION

The aforementioned copending application Ser. No. 09/376,282 describes an embodiment of the invention therein which is directed to a generally T-shaped brief, e.g., a diaper which comprises a chassis having two intersecting portions which together define a generally T-shaped configuration when the chassis is laid out flat and viewed in stretched position. One of the two portions or pieces is a crosspiece comprising opposed lateral segments or wings adapted to be wrapped around the waist of a wearer of the diaper such as to overlap each other, and the second portion or piece is vertical relative to the crosspiece and has a proximal end, and a distal end which can be passed under the crotch and folded upwardly and over the overlapped wings. The T-shaped diaper described in said copending application is provided with a fastening system designed to secure the diaper tightly but comfortably around the waist as illustrated in FIGS. 23-28 of said application. Another variant of the invention, i.e., a generally L-shaped diaper is shown in FIG. 29. The disclosure of said application Ser. No. 09/376,382 is fully incorporated herein by reference.

T-shaped diapers have received widespread attention and acceptance due, in part, to their relative ease of fabrication and use, and the comfort they provide to the wearer as well as the protection they afford against leakage of urine and body exudates. A variety of T-shaped briefs or diapers have been described in the prior art.

For example, one T-shaped diaper was described in U.S. Pat. No. 4,051,854 issued to Gabrielle Leonie Aaron on Oct. 4, 1977. The diaper described therein has a center flap and two ear flaps which, when laid flat, defines a T-shape configuration as shown in FIGS. 1 and 3 of that patent. Fastening means are provided in the form of multiplicity of hooks and loops for fastening the ear flaps to the center flap.

Another T-shaped diaper is described in U.S. Pat. No. 4,995,873 issued to Jacklyn M. Knight on Feb. 26, 1991 which comprises a crosspiece and an intersecting piece which together define the T-shape configuration of the diaper when laid out in flat position. The crosspiece has opposite ends or wings which encircle the waist in an overlapping manner and the intersecting piece passes under the crotch and upwardly and over the crosspiece. Releasable fastening means such as press-on/rip-off adhesive or Velcro® strips are employed to attach the intersecting piece to the crosspiece.

Whether using a T-shaped diaper or other types of diapers, it is essential that the diaper meet at least two significant criteria, i.e., comfort to the wearer and protection against leakage of body fluids and exudates. In order to achieve these objectives, many prior art workers in the field have focused on providing the diaper with an effective fastening system. Indeed, in the aforementioned application Ser. No. 09/372,382, the inventors describe a fastening system for T-shaped diaper designed to achieve the foregoing objectives. Thus, in the T-shaped diaper described therein, one female fastening means is provided on the lateral wing or segment of the crosspiece and at least one male fastening means is provided on the other lateral wing or segment of the crosspiece such that when the two lateral segments are wrapped around the waist and overlap each other, the male fastener engages the female fastener. In addition, a pair of spaced apart female fasteners are provided on said crosspiece between the fasteners in the wings such that when the two wings are wrapped around the waist and folded over each other as aforesaid, said two spaced apart female fasteners will be engaged by a pair of spaced apart male fasteners located at the distal end of the vertical piece of the T-shaped diaper.

As it can be seen from the description in the aforementioned application Ser. No. 09/376,382 and the prior art in general, a variety of fasteners are employed such as, e.g., adhesive tape tabs, Velcro®, fabrics which act as female surface for a male fastener, so-called hook and loop fasteners, or even mechanical elements. As it can also be appreciated, the concern over providing a leak-proof, and good fitting diaper is not limited to a particular shaped diaper. Regardless of their shapes, it is essential that the diaper be provided with an effective fastening means which assures tight but comfortable wear, and affords protection against leakage of fluid and body exudates.

Recently, in U.S. Pat. No. 5,906,604 issued on May 25, 1999 to Ronnberg et al., the patentee describes an attachment means for a belt used with an absorbent garment. The belt is either integrated with the absorbent garment, or it can be a separate belt attached to the absorbent garment by means of a releasable attachment such as hook and loop type fastening means, e.g., VELCRO®. Other fastening systems are described in the patents referred to in the aforementioned Ronnberg et al. patent as well as a host of other prior art patents.

A mechanical fastening system for absorbent articles is described in U.S. Pat. No. 5,279,604 issued to Robinson et al. on Jan. 18, 1994. The mechanical fastening system described therein comprises a tape tab having a first fastening element, a landing member comprising a second fastening element which is engageable with the first fastening element, and an additional fastening element for securing the absorbent article in a manner which facilitates disposal of the article.

Notwithstanding the plethora of prior art patents describing a variety of fastening systems for different diapers, there is still a need for a fastening system which is inexpensive, simple to apply to the garment and is effective in preventing leakage of body fluids and exudates from the diaper.

Accordingly, it is an object of the present invention to provide an absorbent article, e.g., a diaper, which has an improved fastening system.

It is also an object of this invention to provide such diaper with a fastening system which is easy to apply to the diaper and which is adjustable to assure comfort and fitness to the body of the wearer, and which can afford maximum protection against leakage of urine and body exudates.

It is another object of this invention to provide a diaper having an improved fastening system wherein the diaper, when laid out flat, has a T-shaped configuration.

The foregoing and other features and advantages of the present invention will be appreciated from the following detailed description taken with the accompanying drawings.

### SUMMARY OF THE INVENTION

The present invention provides a diaper, such as a T-shaped diaper, having novel fastening system. The T-shaped diaper has a chassis comprising two intersecting portions or cross pieces which define the T-shape configuration of the diaper when viewed in stretched position. One portion is a lateral piece having lateral segments or wings which are adapted to be wrapped around the waist of a wearer of the diaper. The second portion is a vertical piece having a proximal end attached to the chassis and a distal end with tape tabs. The vertical portion is adapted to be passed under the crotch region of the diaper, folded thereover and attached to landing zones on the surface of the lateral segments by the tape tabs located at said distal end.

In order to assure fit and comfort, a novel tape tab is provided at or near one of said segments or wings, e.g., the right segment when the diaper is viewed in front stretched position. Tape tabs are also provided at the distal ends of the intersecting vertical portion which may be similar to the tape tab located on the wing of the diaper.

In accordance with this invention several fastening systems are disclosed which are more fully discussed in the detailed description of the present invention.

### BRIEF DESCRIPTION OF THE DRAWINGS

In the drawings, wherein like reference numerals are employed to designate like parts wherever possible:

FIG. 1 is a perspective view of a T-shaped diaper in assembled form when worn by a wearer;

FIG. 2 is perspective view of the diaper in FIG. 1 showing the center vertical segment with its distal end partly folded upward and away from the diaper chassis;

FIG. 3 is another perspective view of the diaper of FIG. 2 but showing the center vertical segment hanging more freely prior to assembling the diaper;

FIG. 4 is a stretched view of the diaper of FIG. 1 when it is laid out flat;

FIG. 5 is a sectional view taken along the line 5-5 of FIG. 4;

FIG. 6 is a view of a T-shaped diaper in stretched position employing a tape tab and landing zone fastening system according to one embodiment of the present invention;

FIG. 7 is a top view of the tape tab and landing zone fastening system shown in FIG. 6, with the fastener tape in open, ready to be used position;

FIG. 8 is a sectional view taken along the line 8-8 of FIG. 7;

FIG. 9 is a sectional view taken along the line 9-9 of FIG. 6;

FIG. 10 is a partly perspective view of a diaper having a tape tab and landing zone fastening system ready for use;

FIG. 11 is a perspective view of the diaper shown in FIG. 10 in assembled position;

FIG. 12 is a view similar to FIG. 6 employing multi-layer tape tab and landing zone fastening system according to another embodiment of the present invention;

FIG. 13 is a top view of the multi-layer tape tab and landing zone fastening system of FIG. 12 with the fastener tape in open ready to use position;

FIG. 14 is a sectional view taken along the line 13-13 of FIG. 13;

FIG. 15 is a sectional view taken along the lines 15-15 of FIG. 12;

FIG. 16 is a stretched view of a T-shaped diaper similar to FIGS. 11 and 12 but employing a hook and loop fastening system according to a different embodiment of the present invention;

FIG. 17 is a top view of the hook and loop fastening system employed in FIG. 16 with the tape tab in open ready to be used position;

FIG. 18 is a sectional view taken along the line 18-18 of FIG. 17;

FIG. 19 is a sectional view taken along the line 19-19 in FIG. 16;

FIG. 20 is a view similar to FIG. 16 but employs a multi-layer hook and loop fastening system according to yet another embodiment of present invention;

FIG. 21 is a top view of the multi-layer hook and loop fastening system used in FIG. 20 with the tape tab in open ready to be used position;

FIG. 22 is a sectional view taken along the line 22-22 in FIG. 21;

FIG. 23 is a sectional view taken along the line 23-23 in FIG. 20;

FIG. 24 is a view similar to FIG. 10 employing a multi-layer hook and loop fastening system.

FIG. 25 is a view similar to FIG. 20 wherein the diaper is partly elasticated;

FIG. 26 is a sectional view taken along the line 26-26 of FIG. 25;

FIG. 27 is a sectional view taken along the line 27-27 of FIG. 25;

FIG. 28 is a view similar to FIG. 16 with the distal end of the vertical section having laterally extended segments or wings;

FIG. 29 is a view similar to FIG. 20 with a male fastener at one wing and a female fastener is provided at the opposite wing;

FIG. 30 is a sectional view taken along the line 30-30 of FIG. 29;

FIG. 31 is a sectional view taken along the line 31-31 of FIG. 29;

FIG. 32 is a sectional view taken along the line 32-32 of FIG. 29;

FIG. 33 is a sectional view taken along the line 33-33 of FIG. 29;

FIG. 34 is a view similar to FIG. 16 having different tape tab positions at the distal end of the vertical portion;

FIG. 35 is a sectional view taken along the lines 35-35 of FIG. 34;

FIG. 36 is a stretched view similar to FIG. 25 wherein each way segment is folded over itself; and

FIG. 37 is a sectional view taken along the line 37-37 of FIG. 36.

### DETAILED DESCRIPTION OF THE DIFFERENT EMBODIMENTS OF THE INVENTION

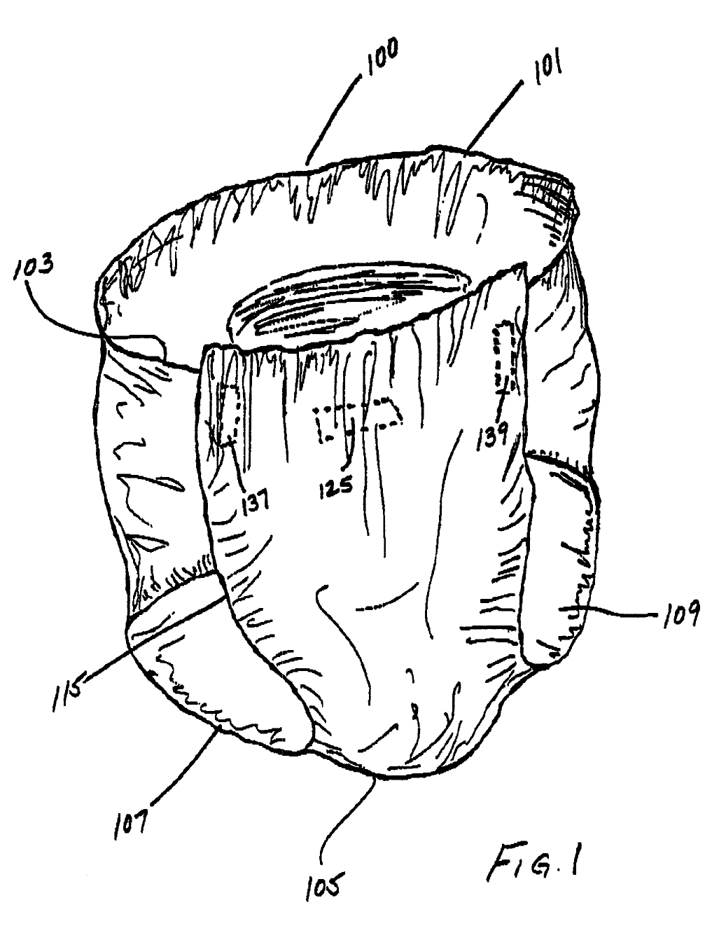

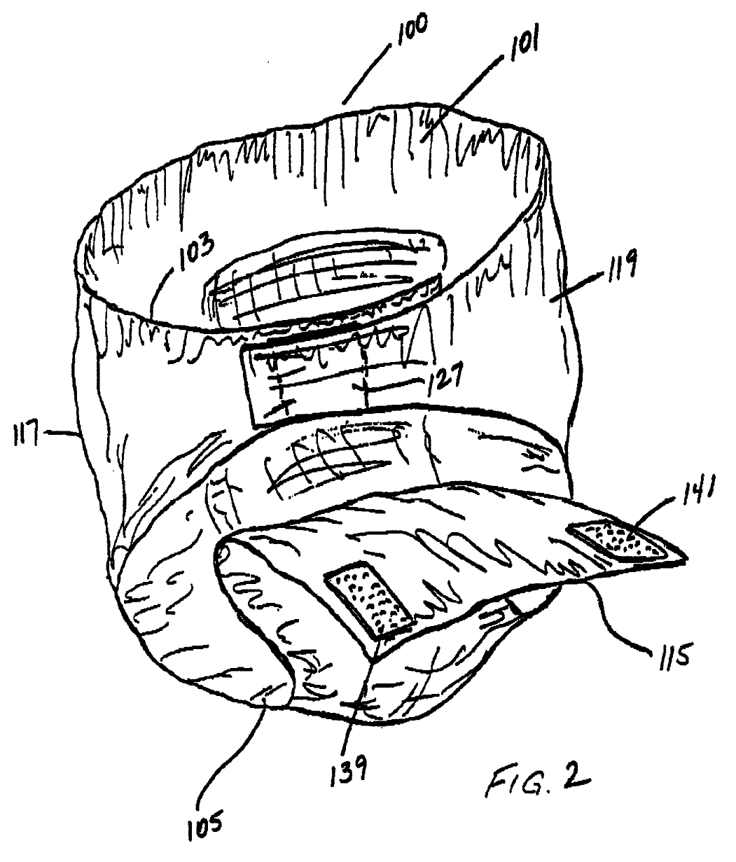

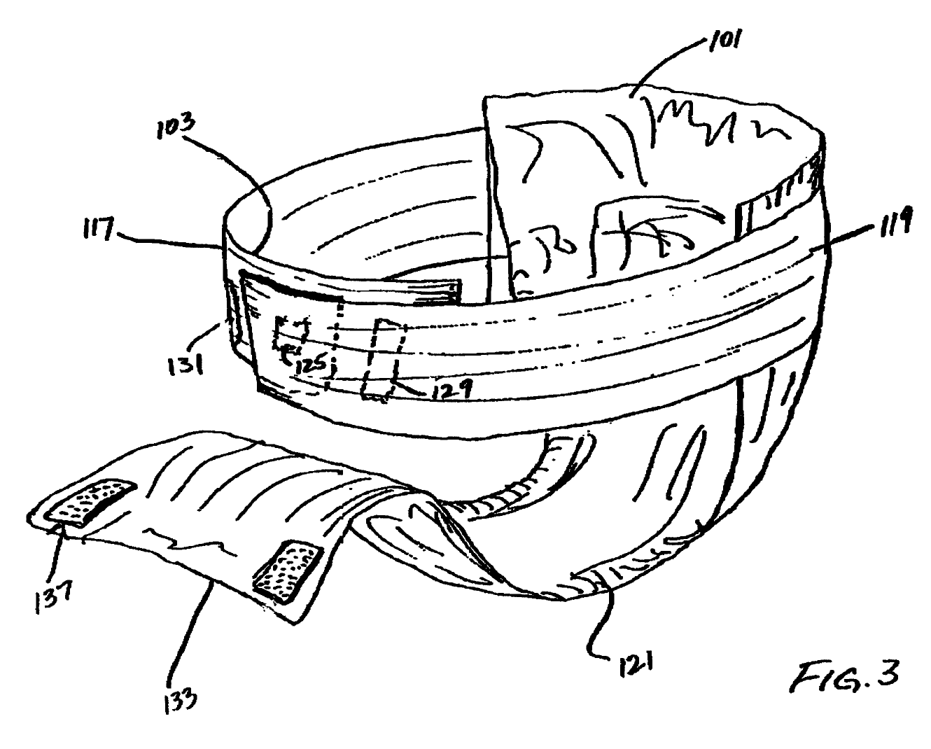

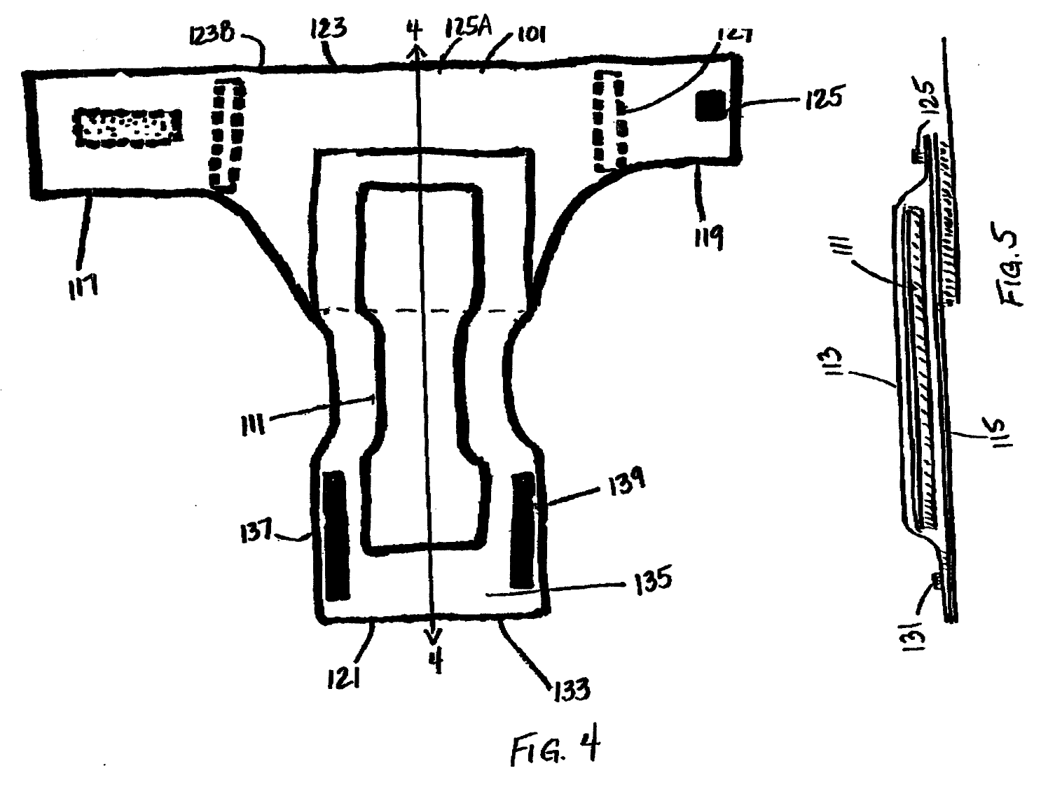

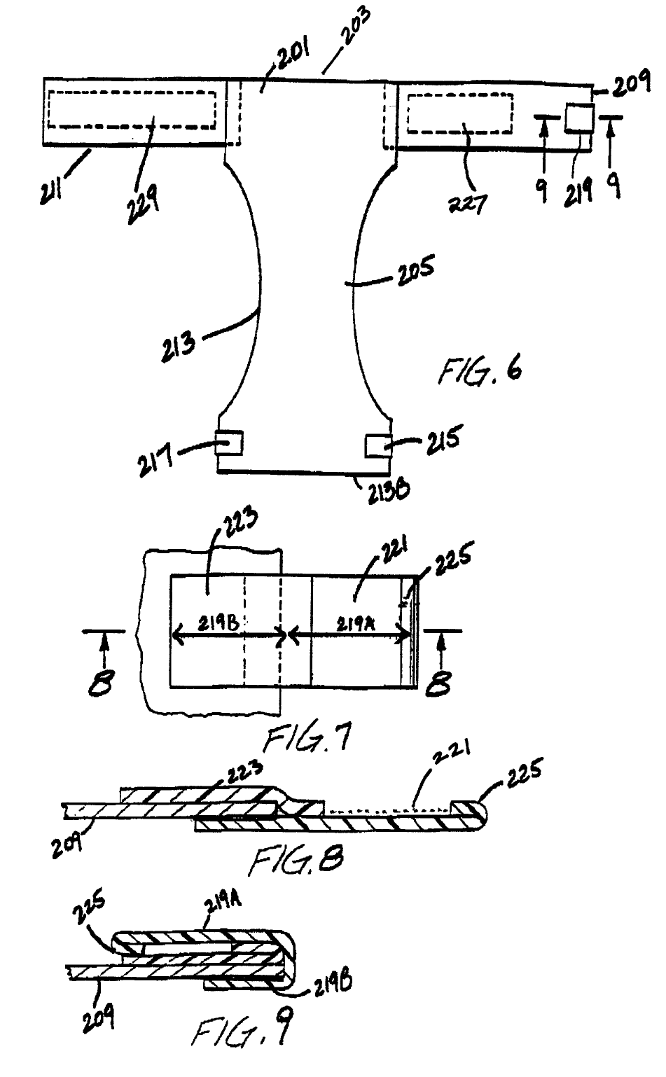

Referring first to FIGS. 1-5, there is shown in FIG. 1, a diaper generally designated as 100 comprising a chassis having a back waist region 101 and a front waist region 103 (which may be elasticized), a crotch region 105 and a pair of leg openings 107, 109 through which extend the legs of the wearer. The diaper 100 also comprises an absorbent core or pad 111 which is disposed between a liquid pervious cover sheet 113 and a liquid impervious backsheet 115 as illustrated in FIG. 5 and described hereinafter. The back waist region 101 comprises a mid region 123 having a pair of opposed laterally extending segments or ears 117, 119 and a center intersecting segment or flap 121 which extends vertically downward relative to the ears 117, 119 as illustrated in FIGS. 2, 3 and 4. The ear segments 117, 119 are adapted to be wrapped around the waist, and the center vertical segment 121 is adapted to be passed under the crotch region 105, pulled up and folded over the crotch region and engaged onto the ear segments 117, 119 as hereinafter described. In order to assure a more perfect fit of the diaper around the torso of the wearer, the ear segments 117, 119 and the center flap 121 are provided with fastening means at strategic locations on their respective surfaces. For example, in the embodiment shown in FIG. 4, the front surface 123A of the mid region 123 is provided with a male Velcro-type fastening means 125 adjacent the right lateral end of the ear segments 119, and the reverse surface 123B opposite the surface 123A is provided with a female Velcro-type fastening means 127, 129, 131. The distal end 133 of the center segment 121 has a top surface on which is provided a pair of opposed, generally parallel and spaced apart male Velcro-type fastening means 137, 139. As it will become apparent from the description of the assembly of the diaper during its use, the fastening means on the ear segments and the center segment are located at such positions as to result in a remarkable tight fit diaper which is highly effective against leakage of fluid and body exudates.

In use, the ear segment 117 is wrapped around the waist first, followed by wrapping the ear segment 119 around the waist to overlap the ear segment 117, and the male Velcro fastener 125 is engaged onto the female Velcro fastener 131. The center segment 121 is then passed under the crotch of the wearer and folded thereover and upwardly onto the top surface 123A of the diaper, and then engaging the male Velcro fasteners 137 and 139 onto the corresponding aligned female Velcro fasteners 127 and 129 respectively. By proper spacings and alignments of the male/female Velcro fasteners, the fasteners will mate, i.e., inter-engage, thus resulting in a more perfect fit to the waist of the wearer and prevent leakage of the body fluids and exudates out of the diaper.

The materials and fabrics used in making the diapers of the present invention are generally of the type and variety known in the art and are described, for example, in U.S. Pat. Nos. 4,695,278 and 4,795,454 and in copending, commonly assigned application Ser. No. 09/149,265, filed Sep. 8, 1998, the disclosures of which are fully incorporated herein by reference. Thus, the liquid pervious cover sheet is a compliant soft material which is skin friendly and does not cause rash or irritation. Such materials include porous foams, reticulated foams, plastics, natural fibers such as woods or cotton fibers, synthetic fibers made of polyester or propylene available from First Quality Fibers, Inc., McElhattan, Pa., or made from a combination of such materials.

The absorbent pad or core may be manufactured from a wide variety of liquid absorbent materials of the type usually used in manufacturing disposable diapers and other absorbent articles. Such materials include comminuted wood pulp, creped cellulose wadding, absorbent foams and sponges, super absorbent polymers, or a combination of said materials.

The acquisition layer is usually made of chemically bonded nonwoven polypropylene available from American Nonwovens, Columbus, Mo. Preferably, the width of this layer is substantially the same as the width of the absorbent core. This core may be made of wood pulp fibers and super absorbent polymers such as IM 7000 series available from Clarian Products, Inc., Portsmouth, Inc., Va., and Chemdal 200 series, available from Chemdal Inc. Palantine, Ill. Alternatively, the absorbent core may be made of dual layer construction, in which case, the absorbent polymer may be securely positioned between each layer of the absorbent material.

The film backing is usually a polyethylene layer which is liquid, air and preferably vapor impermeable, and is placed under the absorbent core to prevent the body exudates from leaking and otherwise soiling the user's bed and clothing. The width and length of the backing film are generally at least equal to the width and length of the absorbent core. Polyethylenes suitable as backing film for the purpose of this invention are available from Clopay Plastics, Cincinnati, Ohio. The backsheet is also preferably made of spunbond nonwoven polypropylene and is usually coextensive with the backing film. In general, however, the various layers are of the type and materials well known in the diaper industry and within the scope and knowledge of those versed in this art.

In the T-shaped diaper described and illustrated by reference to FIGS. 1-5, the fastening system employed is the system described in the aforementioned application Ser. No. 09/376,282, the disclosure of which is fully incorporated herein by reference. The ensuing description, however, will be directed to the novel fastening system of the present invention, and will be described in connection with a T-shaped diaper, although it may be employed in other absorbent articles as well.

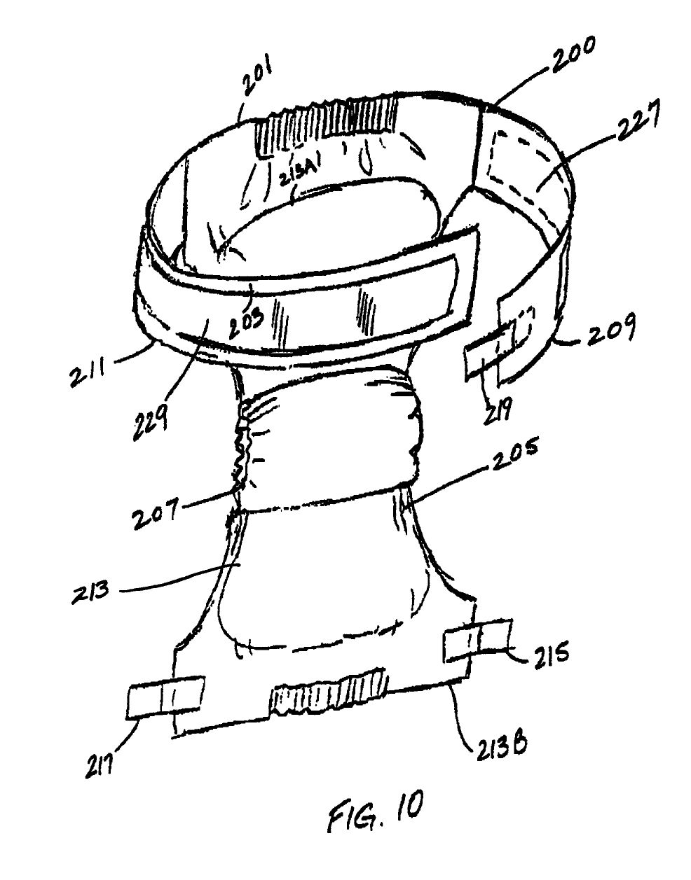

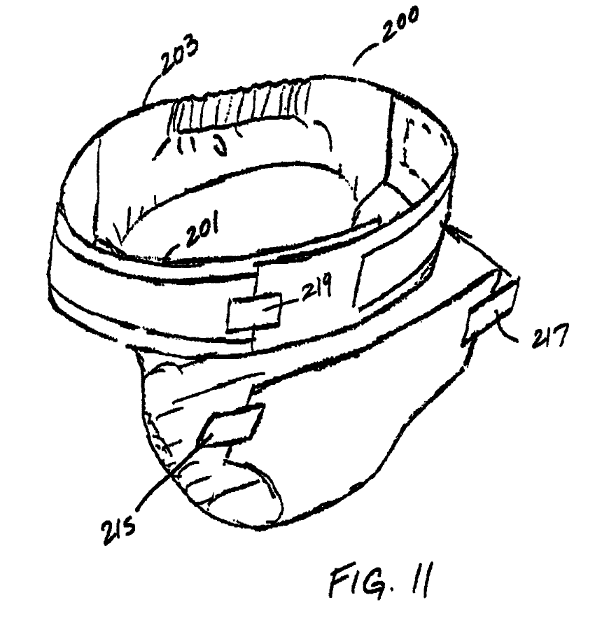

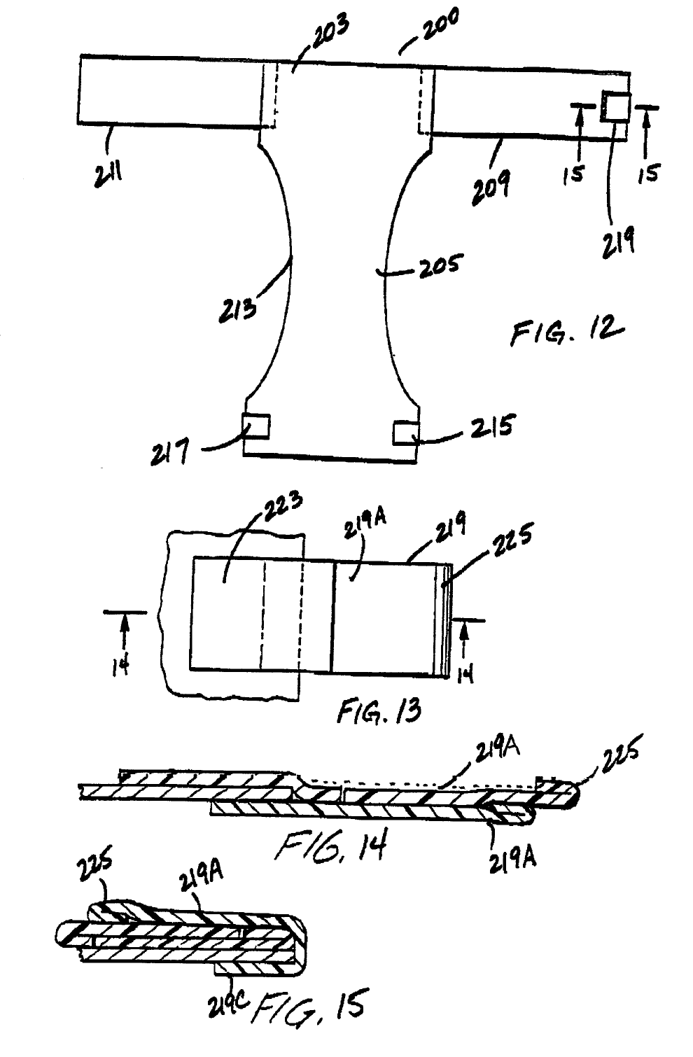

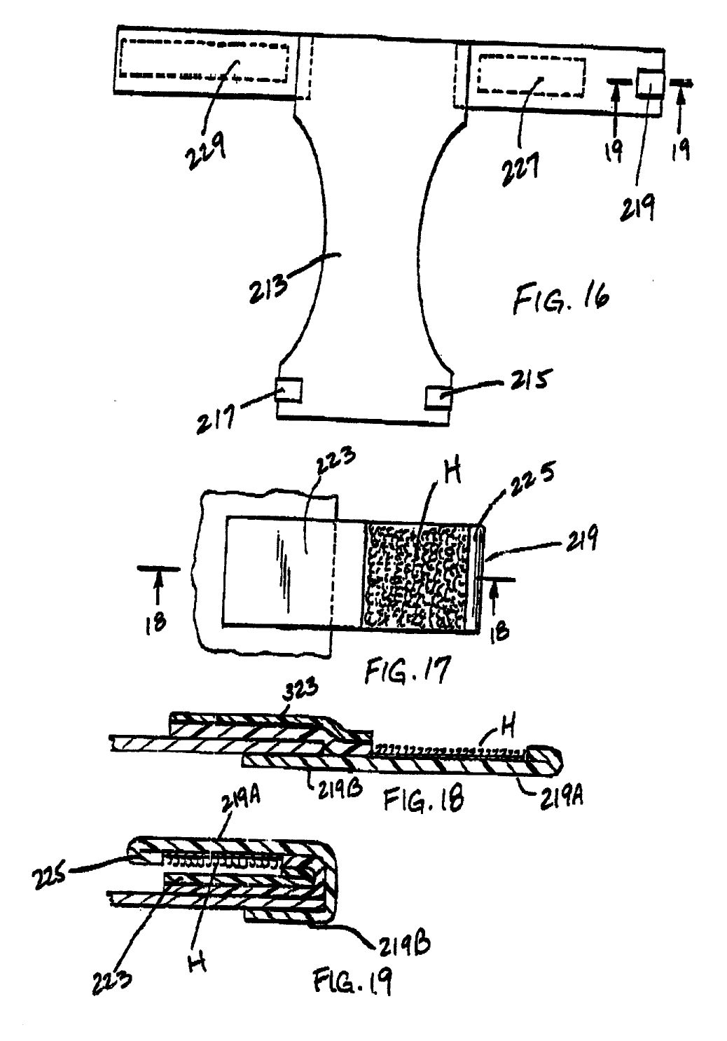

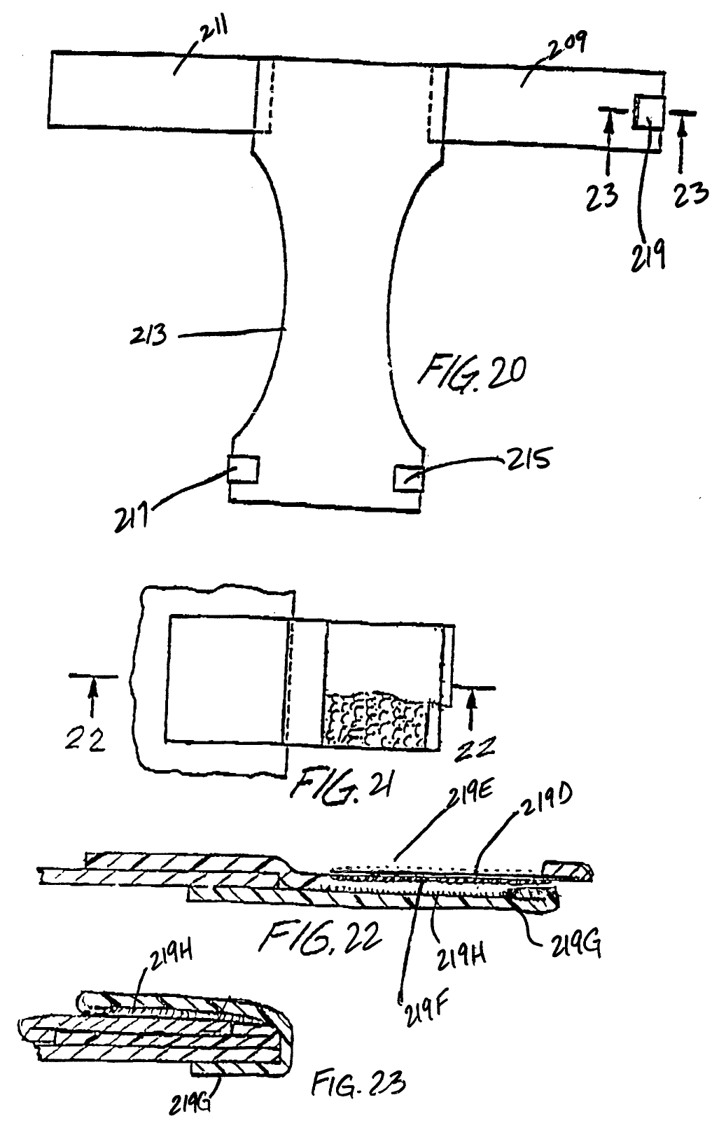

A first embodiment of the fastening system of the present invention is shown in FIGS. 6-9 and a diaper incorporating this fastening system is shown, in perspective view, in FIGS. 10-11. As shown in FIGS. 10 and 11, the diaper generally designated as 200 comprises a chassis having a back waist region 201 a front waist region 203 and a crotch region 205 and an absorbent core 207. A pair of opposed lateral segments or wings 209, 211 extend from the respective edges of the back waist region 201, and a generally vertical intersecting piece 213 having a proximal end 213A attached to the diaper chassis and a distal end 213B with a pair of opposed tapes 215, 217 disposed at each side of the intersecting piece near the distal end thereof.

Referring to FIGS. 6-9, there is shown in FIGS. 6 and 7 the tape tab 219 having a portion 219A used to fasten the tape to the diaper, and a second portion 219B permanently attached to the bottom surface of the wing 209, i.e., the surface away from the skin of the wearer (see FIGS. 8 and 9). The tape portion 219 has a top surface 221 covered with a pressure sensitive adhesive, and an opposed bottom surface made of a suitable plastic such as, e.g., polyethylene or polypropylene film, or other material such as, e.g., woven or nonwoven. As shown in FIG. 7, the fastening system of this embodiment comprises a release tape 223 having a top surface coated with a release agent such as a silicone compound, and an opposed bottom adhesive surface with a portion of the release tape 223 attached to the portion 219A and the other portion adhered to the top surface of the wing 209. Before use, the portion 219A is folded over the portion 219B in order to protect the adhesive surface during transportation of the product. For convenience of manipulation, the lateral edge of the tape 219 is folded upon itself so as to form an adhesive-free grip strip (Finger lift) 225.

The other component of the fastening system in this embodiment of the invention are landing zones (tapes) 227, 229 located on the lateral segments or wings 209 and 211, respectively. The size of each landing zone may be varied if desired depending on the size of the diaper. Each landing zone has an outer surface covered, at least partly, with a release agent such as a silicone compound. This allows the tape tab 219 to be positioned and repositioned on the landing zone 229 several times without tearing the diaper fabric.

In use, the tape tabs 219 is peeled away from the tape tab 219A by gripping and pulling away the grip strip 225. The diaper wings 209 and 211 are then wrapped around the waist of the wearer and the pressure sensitive surface 221 of the tape 219 is secured to the landing zone 229. Thereafter, the insert piece 213 is passed under the crotch, folded thereover and the tape tabs 215 and 217 located at the distal end of the insert piece is releasably secured to the landing zones such that tape tab 215 is secured to the landing zone 227 and tape tab 217 is secured to the landing zone 229. Tape tabs 215 and 217 may each have the same construction as tape tab 219, if desired. As it can be seen, the fastening system permits repeated adjustments and repositioning of the tape tabs on the landing zones to achieve a desired fit without tearing the fabric of the diaper.

The second embodiment of the present invention defines of multi-layer tape tabs and landing zone shown in FIGS. 12-15 which is similar to the first embodiment illustrated in FIGS. 6-9 except for differences in the fastener 219 discussed below. As shown in FIG. 12 the T-shaped 200 comprises a chassis having opposed lateral segments or wing 209, 211. The construction of the diaper is otherwise the same as in FIG. 6. The fastener 219, however, consists of two layers of tapes 219A, 219C. Initially, the layer 219A is used to fasten the tape 219 to the diaper. In order to readjust the diaper, the layer 219A may be peeled off, leaving the layer 219C in place to act as a landing zone for readjustment of the diaper when necessary. After readjusting the diaper, the layer 219A is attached back onto layer 219C. Thus, the second fastening system requires less landing zone than the first embodiment while still realizing the advantage of repeated readjustment of the diaper and repositioning of the tape tab without tearing the diaper fabric. The use of multi-layer tape tab according to the second embodiment, with less landing zone area, results in increased flexibility of the wing portion of the diaper, and permits the use of elasticized wings when desired, all resulting in decreased manufacturing cost of the diaper. The construction of tape tabs 215, 217 may be similar to tape tab 219.

The third embodiment of the present invention employs hook and loop fastening system as illustrated in FIGS. 16-19. This fastening system is similar to the first fastening system described in connection with FIGS. 6-9 except that the landing zones 227 and 229 is covered with loop material rather than a silicone compound and the top surface of the tape portion 219A is covered with a hook material which covers the pressure sensitive adhesive. Thus, the release tab 223 covers the adhesive area that is not covered by the hook material. Referring to FIGS. 16-19, it can be seen that the configuration and construction of the T-shaped diaper of FIG. 16 is the same as in FIG. 6. Instead of being covered by a pressure sensitive adhesive as in FIG. 7, the portion 219A is covered with layer of hook material H, e.g., Velcro®. A grip strip 225 facilitates gripping the end of the tape 219 when peeling the fastener.

In use, the diaper wings are wrapped as hereinbefore described, the grip strip 225 is gripped to peel the tape 219 away and expose the hook surface H and then attaching (engaging) the hook surface H onto the hook landing zones 227, 229 on the wing. The intersecting portion 213 is then passed under the crotch, folded thereover and the tabs 215, 217 are secured to the landing zones 227, 229, respectively. Again the construction of tape tabs 215, 217 may be identical to tape tabs 219 in FIGS. 6, 12 and 16.

The fourth embodiment of the present invention is similar to the second embodiment except that the tape 219 consists of two superposed layers; a layer 219D having a top adhesive surface 219E and an opposed bottom surface 219F covered with loop material. Superposed on the tape 219D is the layer 219G which is covered with hook material H, the same as layer 219A in FIG. 18.

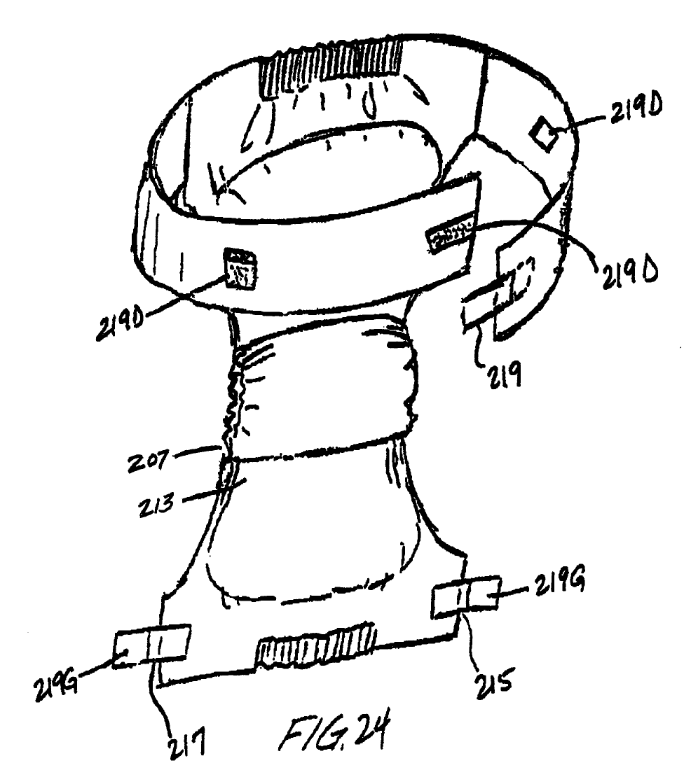

FIG. 24 illustrates a diaper, in semi-assembled position, incorporating the fastening system described in the second and fourth embodiment of the present invention. In FIG. 24, the tabs 215, 217 are shown in open position after use of the diaper in order to further illustrate the fastening system. As it can be seem from this figure, after the diaper has been worn and the insert piece 213 has been removed to disassemble the diaper, there remains on the wings 227 and 229, the layers 219D as described in connection with FIG. 22.

From the foregoing detailed description it is evident that several changes and modifications may be made in the different fastening systems which are obvious from, and are suggested by the description herein. It must also be noted that the nature of the different layers, the hook and loop materials and the adhesives used are well known in the art and are mentioned in the prior art patents discussed in this application as well as the earlier related patent applications.

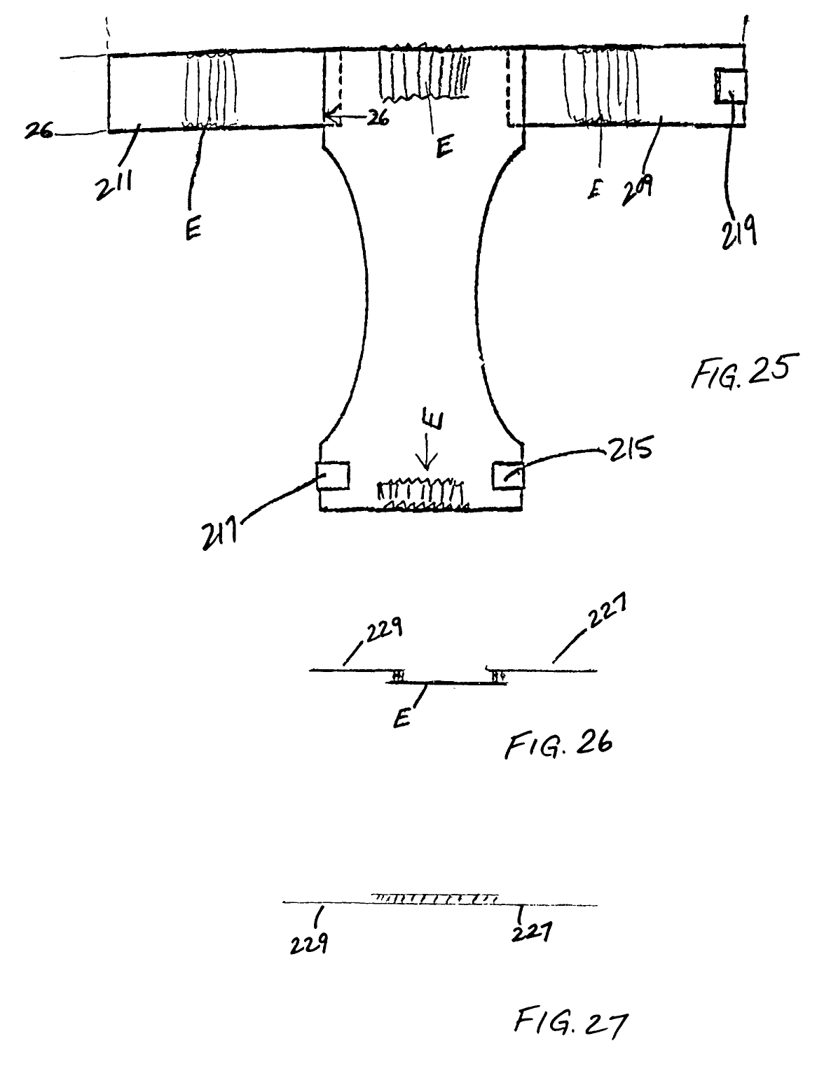

Referring to FIG. 25, the diaper shown therein has elasticated portions E attached to the wings, the mid-waist section and the distal end of the insert 213 or vertical intersecting portion. The elastic material may be any of the known elastic materials used in the art. This inclusion of elastics in the aforementioned diaper portion adds to stretchability of the wings and the diaper for improved fit and comfort. Also, the wings dimension, i.e., length L and width W can vary depending on the desired size. The diaper wings are usually made from spunbond, spunbond and meltblown, thermally or chemically bonded, hydrogenated nonwoven, composites made of film and nonwoven laminates and composites made of nonwoven/film/nonwoven. The film may be elastic and the nonwoven may have 50 to 200 percent elongation capability. If desired, the composite may be apertured in order to further improve its stretchability. The wings may be added as separate pieces on each side of the chassis insert, or they can be formed as extension of the insert.

The insert 213 used in the diaper consists of a fluid permeable coversheet, a fluid impermeable backsheet and an absorbent pad sandwiched therebetween. The absorbent pad may be made of a superabsorbent polymer (SAP) of the type used in the art and wood pulp fibers having the desired density. The ratio of SAP to wood pulp may be varied over a wide range. If desired, a layer or multilayer of drylaid type material can be used as the absorbent pad, such as Rayoner Novathin 2250355 or 3400355 available from Rayonier. The front and backwaist of the insert may be elasticized by attaching elastic web between the coversheet and the backsheet of the insert front and waist area similar to the wings.

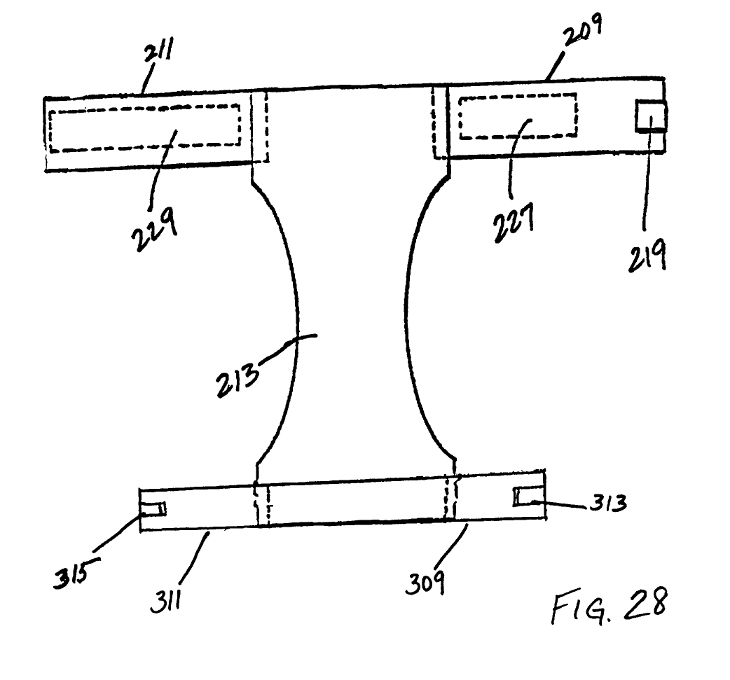

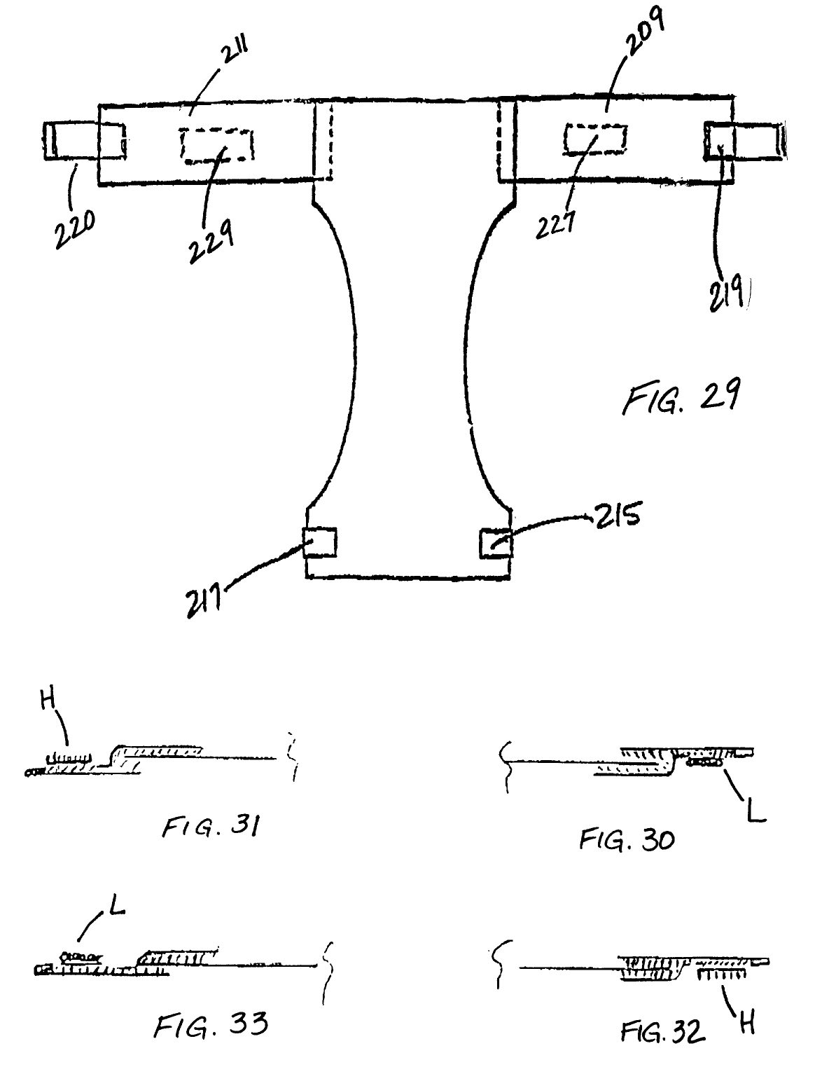

FIG. 28 illustrates an embodiment similar to the embodiment of the invention illustrated by FIG. 16. However, the embodiment shown in FIG. 28, the distal end of the vertical insert piece has lateral segments or wings 309, 311. Male fastener 313, 315 are located at or near the lateral ends of each of the wings 309, 311.

In the diaper shown in FIG. 29, one wing has a male fastener as in FIG. 20 and the opposed lateral wing 211 has a female fastener 220. In addition, a female fastener 227, 229 are attached each on the back of each wing (the side away from the skin of the wearer).

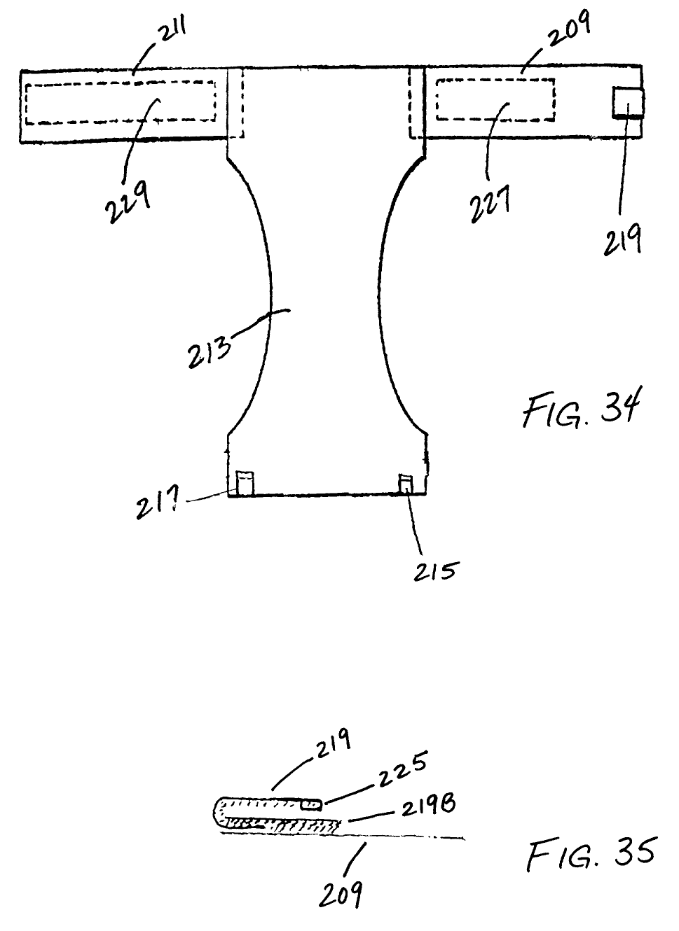

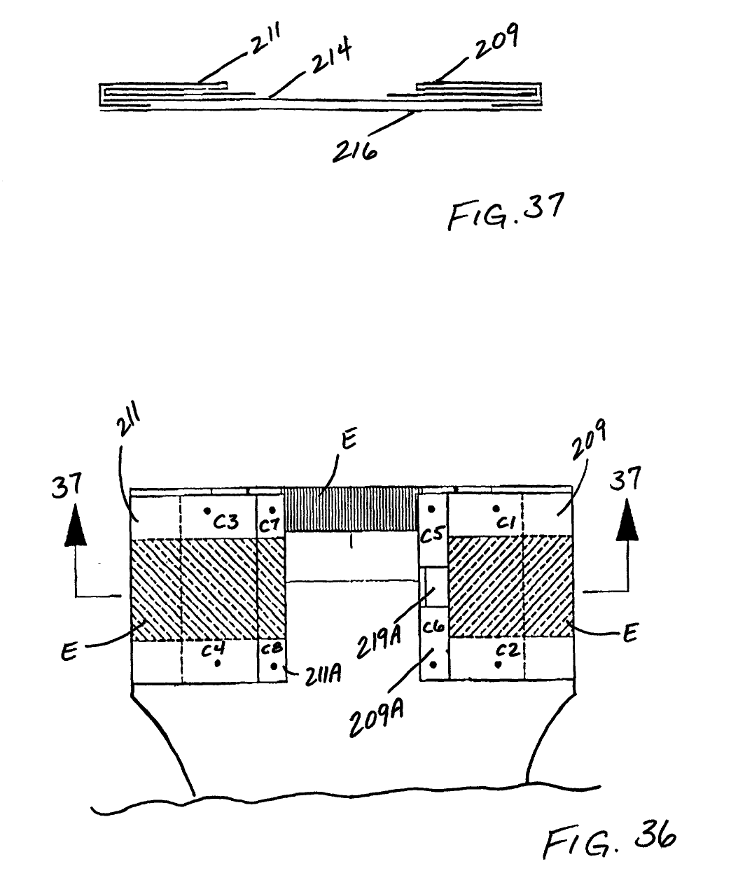

In the diaper shown in FIG. 34, female fasteners 227, 229 are attached to the back surface of the diaper wings 209, 211. The tape tabs 215, 217 and 219 differ from the tape tabs shown in FIGS. 6-9 in that the permanent portion 219B is eliminated in order to reduce the cost of the product (see FIG. 35).

In order to facilitate packaging the diaper, and as shown in FIG. 36, each of the lateral segment or wing 209, 211 is folded upon itself toward the center of the chassis. The facing surfaces of each wing may be attached adhesively, ultrasonically or by some other suitable attachment means as indicated by dotted circles C1, C2, C3 and C4. As it can be seen from FIG. 36, a portion of each wing 209, 211 also cover the insert cover sheet and is similarly attached as shown by the dotted circles C5, C6, C7 and C8. After unpacking, and in order to use the diaper, the finger lift portion 209A, 211A on each wing may be gripped between the thumb and the forefinger and the wing is lifted to an open position.

FIG. 27 shows the composition of the chassis beneath the wings 209, 211 comprising a layer of nonwoven (insert coversheet) 214 and a film backing (insert backsheet) 216.

1. An absorbent article having a chassis comprising two portions which together define a generally T-shaped configuration when said chassis is viewed in stretched position; a first portion having opposed lateral segments adapted to be wrapped around the waist of a wearer of said article, a second portion having a distal end and a proximal end disposed generally vertically relative to said first portion and adapted to be passed under the crotch of the wearer and folded upwardly and over said overlapped portions;
one male fastening means on one of said lateral segments, and at least one female fastening means on said lateral segment, such that when said lateral segments overlap each other said male fastening means engages said female fastening means;
a pair of opposed spaced apart male fastening means at the distal end of said second portion, and a pair of spaced apart opposed female fastening means at said first portion such when said lateral segments are wrapped around the waist of a wearer and said second portion is folded over said segments, each of said pair of male fastening means engage a correspondingly aligned one of said pair of female fastening means.

2. A generally T-shaped absorbent article as in claim 1 wherein at least one of said lateral segments has a female fastening surface adapted to be engaged into the male fastening means.

3. An absorbent article as in claim 1 wherein said second portion is shorter in length relative to said first portion.

4. An absorbent article as in claim 2 wherein said second portion is shorter in length relative to said first portion.

5. An absorbent article as in claim 1 wherein said first portion is at least partly elasticated.

6. An absorbent article as in claim 2 wherein said first portion is at least partly elasticated.

7. An absorbent article as in claim 3 wherein said first portion is at least partly elasticated.

8. An absorbent article as in claim 4 wherein said first portion is at least partly elasticated.

9. An absorbent article having a chassis comprising two portions which together define a generally T-shaped configuration when said chassis is viewed in stretched position; a first portion having a female surface, and a second portion disposed generally vertically relative to said first portion and adapted to be passed under the crotch of the wearer and folded upwardly and over said first portion;
said first portion having opposed lateral segments adapted to be wrapped around the torso of the wearer,
at least one male fastening means on one of said lateral segments,
at least one male fastening means at the distal end of said second portion,
such that when said lateral segments are in overlapped position and said second portion is folded over said overlapped portions, said male fastening means engages said female surface of said first portion.

10. An absorbent article as in claim 9 wherein said second portion is shorter in length relative to said first portion.

11. An absorbent article as in claim 10 wherein said second portion is shorter in length relative to said first portion.

12. An absorbent article as in claim 11, wherein said first portion is at least partly elasticated.

13. An absorbent article as in claim 9 wherein said first portion is at least partly elasticated.

14. An absorbent article as in claim 11 wherein said first portion is at least partly elasticated.

15. An absorbent article having a chassis comprising two portions which together define a generally L-shaped configuration when said chassis is view in stretched position; a first portion and a second portion disposed generally vertically relative to said first portion, said first portion having a laterally extending segment and said second portion having a distal end and a proximal end, and a flared out portion extending from said proximal end disposed in opposed spaced apart relation relative to said laterally extending segment such as to define said generally L-shaped configuration;
at least one male fastening means on said laterally extending segment;
at least three spaced apart female fastening means disposed in spaced apart relations on said first portion;
a pair of opposed and spaced apart male fastening means on said second portion, at the distal end thereof,
such that when said laterally extending segment and said flared out portion are wrapped around the torso of the wearer, said male fastening means on said laterally extending segment engages the female fastening means on said flared out portion, and when said second portion is folded over onto said wrapped around portion, said male fastening means at the distal end of said first portion engage the correspondingly aligned female fastening means.

16. An integral disposable article as in claim 15 wherein said first portion has a female surface adapted to be engaged into said male fastening means when said second portion is folded over said female surface of said first portion.

17. An integral disposable article as in claim 15 wherein said first portion is at least partly elasticated.

18. An integral disposable article as in claim 16 wherein said first portion is at least partly elasticated.

19. An absorbent article having a chassis comprising two portions, a first waist portion and a second insert portion which together define a generally T-shaped configuration when said chassis is viewed in stretched position; said first waist portion having opposed lateral segments adapted to be wrapped around the waist of a wearer of said article, said second portion having a distal end having at least one tape tab, and a proximal end, disposed generally vertically relative to said first waist portion and adapted to be passed under the crotch of the wearer and folded upwardly and over said waist portion such that the tape tab at the distal end of said second portion is adhered to one of said lateral segments;
said chassis further comprising a tape tab disposed near the end of one of said lateral segments, said tape tab having a first portion for fastening said tape tab to said lateral segment, and a second portion permanently attached to a surface of said lateral segment away from the skin of the wearer of said diaper, said first portion having a top adhesive-covered surface and an opposed bottom backing sheet; a release tape having a top surface coated with a release compound and an opposed bottom adhesive surface wherein a portion of said release tape is attached to said first portion of said tape tab, and another portion of said release tape is adhered to a lateral segment of said diaper.

20. An absorbent article as in claim 19 wherein said first portion of said tape tab comprises an adhesive-free grip strip at the outer lateral end of said first portion.

21. An absorbent article as in claim 19 which is at least partly elasticated.

22. An absorbent article as in claim 20 which is at least partly elasticated.

23. An absorbent article as in claim 19 wherein said first portion of said tape tab consists of two layers, a first layer having a top adhesive-coated surface and a second layer having a bottom adhesive surface adhered to said top layer, and wherein said second surface is initially adhered to said first layer but is peelable therefrom before use of the absorbent article.

24. An absorbent article as in claim 23 wherein said first portion of said tape tab comprises an adhesive-free grip strip at the outer lateral end of said first portion.

25. An absorbent article as in claim 23 which is at least partly elasticated.

26. An absorbent article as in claim 24 which is at least partly elasticated.

27. An absorbent article as in claim 19 wherein said top surface of said first portion consists of an adhesive layer and a superposed layer of hook material and wherein the lateral segment to be engaged by said tape tab is at least partly covered with loop material.

28. An absorbent article as in claim 27 wherein said first portion of said tape tab comprises an adhesive-free grip strip at the outer lateral end of said first portion.

29. An absorbent article as in claim 27 which is at least partly elasticated.

30. An absorbent article as in claim 28 which is at least partly elasticated.

31. An absorbent article as in claim 19 wherein said bottom surface of said tape tab consists of two superposed layers, a first layer having a top adhesive covered surface and an opposed bottom surface covered with loop material, and a second layer which is covered with hook material.

32. An absorbent article as in claim 31 wherein said first portion of said tape tab comprises an adhesive-free grip strip at the outer lateral end of said first portion.

33. An absorbent article as in claim 3 which is at least partly elasticated.

34. An absorbent article as in claim 32 which is at least partly elasticated.

35. An absorbent article as in claim 19 wherein a male fastener is attached near the end of one of said lateral segments and a female fastener is attached near the end of the other lateral segments such that when said lateral segments overlap the waist portion, said male fastener engages said female fastener, and wherein said second portion of the chassis comprises at least one male fastener attached at the comers of said distal end of said second portion.

36. An absorbent article as in claim 35 wherein said first portion of said tape comprises an adhesive-free grip at the outer lateral end of said first portion.

37. An absorbent article as in claim 35 which is at least partly elasticated.

38. An absorbent article as in claim 36 which is at least partly elasticated.

39. An absorbent article as in claim 23 wherein a male fastener is attached near the end of one of said lateral segments and a female fastener is attached near the end of the other lateral segments such that when said lateral segments overlap the waist portion, said male fastener engages said female fastener, and wherein said second portion of the chassis comprises at least one male fastener attached at the comers of said distal end of said second portion.

40. An absorbent article as in claim 39 wherein said first portion of said tape tab comprises an adhesive-free grip strip at the outer lateral end of said first portion.

41. An absorbent article as in claim 39 which is at least partly elasticated.

42. An absorbent article as in claim 40 which is at least partly elasticated.

43. An absorbent article as in claim 27 wherein a male fastener is attached near the end of one of said lateral segments and a female fastener is attached near the end of the other lateral segments such that when said lateral segments overlap the waist portion, said male fastener engages said female fastener, and wherein said second portion of the chassis comprises at least one male fastener attached at the comers of said distal end of said second portion.

44. An absorbent article as in claim 43 wherein said first portion of said tape tab comprises an adhesive-free grip strip at the outer lateral end of said first portion.

45. An absorbent article as in claim 43 which is at least partly elasticated.

46. An absorbent article as in claim 44 which is at least partly elasticated.

47. An absorbent article as in claim 31 wherein a male fastener is attached near the end of one of said lateral segments and a female fastener is attached near the end of the other lateral segments such that when said lateral segments overlap the waist portion, said male fastener engages said female fastener, and wherein said second portion of the chassis comprises at least one male fastener attached at the comers of said distal end of said second portion.

48. An absorbent article as in claim 47 wherein said first portion of said tape tab comprises an adhesive-free grip strip at the outer lateral end of said first portion.

49. An absorbent article as in claim 47 which is at least partly elasticated.

50. An absorbent article as in claim 48 which is at least partly elasticated.

51. An absorbent article as in claim 19 wherein the distal end of said second portion has opposed laterally extending wings which, together with said first waist portion, define a generally H-shaped configuration when said absorbent article is viewed in stretched position.

52. An absorbent article as in claim 23 wherein the distal end of said second portion has opposed laterally extending wings which, together with said first waist portion, define a generally H-shaped configuration when said absorbent article is viewed in stretched position.

53. An absorbent article as in claim 27 wherein the distal end of said second portion has opposed laterally extending wings which, together with said first waist portion, define a generally H-shaped configuration when said absorbent article is viewed in stretched position.

54. An absorbent article as in claim 31 wherein the distal end of said second portion has opposed laterally extending wings which, together with said first waist portion, define a generally H-shaped configuration when said absorbent article is viewed in stretched position.

55. An absorbent article having a chassis comprising two portions which together define a generally T-shaped configuration when said chassis is viewed in stretched position; a first waist portion having a female surface, and a second portion disposed generally vertically relative to said first waist portion and having a distal end adapted to be passed under the crotch of a wearer and folded upwardly and over said first waist portion;
said first waist portion having lateral segments adapted to be wrapped around the torso of the wearer;
at least one tape tab on one of said lateral segments;
a pair of opposed spaced apart tape tabs located at said distal end of said second portion;
such that when said lateral segments overlap the waist position and said second portion is folded over said overlapped portions, said tape tabs engage said female surface.

56. An absorbent article having a chassis comprising two portions which together define a generally T-shaped configuration when said chassis is viewed in stretched position; a first portion having a female surface, and a second portion disposed generally vertically relative to said first portion and adapted to be passed under the crotch of the wearer and folded upwardly and over said first portion;
said first portion having opposed lateral segments adapted to be wrapped around the torso of the wearer,
at least one male fastening means on one of said lateral segments,
at least one male fastening means at the distal end of said second portion,
wherein each of said lateral segments is folded upon itself toward the center of such chassis and each of said folded segment comprises a portion adapted to be gripped and lifted to thereby lift said segment to an open position.
